(19) United States
(12) Patent Application Publication
(10) Pub. No.: US 2024/0326687 A1
(43) Pub. Date: Oct. 3, 2024

(54) VEHICLE-MOUNTED DEVICE

(71) Applicant: Kai CHEN, Nanshan Dist (CN)

(72) Inventors: Kai CHEN, Nanshan Dist. (CN); Fan YANG, Nanshan Dist. (CN)

(21) Appl. No.: 18/473,150

(22) Filed: Sep. 22, 2023

(30) Foreign Application Priority Data
Mar. 29, 2023 (CN) 202320642683.5
May 22, 2023 (CN) 202321236266.7
May 22, 2023 (CN) 202321236272.2
Jun. 29, 2023 (CN) 202321668704.7

(51) Int. Cl. B60Q 3/70; A61M 21/02; B60Q 3/20; B60Q 3/50; B60Q 3/60; B60Q 3/80; F21V 21/02; F21V 33/00

(52) U.S. Cl. CPC B60Q 3/70; A61M 21/02; B60Q 3/20; B60Q 3/50; B60Q 3/60; B60Q 3/80; F21V 21/02; F21V 33/0004; A61L 2209/133; A61L 2209/15; A61M 2021/0016

(57) ABSTRACT

The present application provides a vehicle-mounted device, including a housing and a decoration assembly; the decoration assembly includes a light-emitting device and a decoration member, the decoration member is detachably provided on the housing, and the light-emitting device is provided outside the housing. Both the light-emitting device and a cartoon device are provided on the housing of the vehicle-mounted device, so that esthetic experience of a user can be increased, and the vehicle-mounted device is more esthetically pleasing when being decorated in the environment inside a vehicle. The light-emitting device is provided as a sound-sensitive lamp, so that the light-emitting effects are converted according to the sound in the vehicle, the use effect is better and the interestingness is stronger.

### TECHNICAL FIELD

The present application relates to the technical field of automobile decoration, and in particular, to a vehicle-mounted device.

### BACKGROUND

With the popularization of automobiles, private automobiles have become a common tool in people's daily lives. In order to increase the comfort of use, many people often choose to place cartoon decorations, aromatherapy decorations, and other articles inside the automobile to optimize the environment inside the automobile. However, there are the following problems about the existing vehicle-mounted accessories: the existing vehicle-mounted accessories have poor decorative effects and have a single function. There is an urgent need for a vehicle-mounted aromatherapy decoration that integrates decoration and other functions.

### SUMMARY

Based on this, it is necessary to provide a vehicle-mounted device, including a housing and a decoration assembly; the decoration assembly includes a light-emitting device and a decoration member, the decoration member is detachably provided on the housing, and the light-emitting device is provided outside the housing.

Further, the vehicle-mounted device further includes an aromatherapy structure, and the aromatherapy structure is replaceably provided on the housing.

Further, the vehicle-mounted device further includes a power supply, and the power supply is replaceably provided in the housing and supplies power to the light-emitting device.

Further, the decoration assembly further includes a control assembly, both the control assembly and the power supply are electrically connected to the light-emitting device, and the control assembly is provided on the housing and controls the light-emitting device to emit light.

Further, the vehicle-mounted device further includes a fixing device, and the fixing device is provided on one side of the housing.

Further, the fixing device includes a fixing member and a protective member, the fixing member is provided on the housing, and the protective member is provided on the fixing member.

Further, the fixing member is any one of a pin, a clamping claw, and a magnetic fixing device.

Further, the aromatherapy structure includes an aromatherapy frame and an aromatherapy member, and the aromatherapy member is mounted on the aromatherapy frame.

Further, the vehicle-mounted device further includes a quick-release mechanism, and the quick-release mechanism is detachably connected to the housing.

Further, the aromatherapy structure is mounted on the quick-release mechanism.

Further, the decoration member is mounted on the quick-release mechanism.

Further, the housing includes a first portion and a second portion, the second portion is detachably mounted on the first portion, the decoration assembly includes a light-emitting device and a decoration member, the light-emitting device is provided on the first portion, and the decoration member is provided on the second portion.

Further, the vehicle-mounted device further includes an accommodating space, and the accommodating space is provided on the housing or the decoration member.

Further, the vehicle-mounted device further includes an accommodating space, and the accommodating space is provided on the first portion or the second portion.

Further, the vehicle-mounted device further includes a ventilation channel, and the ventilation channel is provided on the housing and connects the accommodating space to an external environment.

Further, the vehicle-mounted device further includes an aromatherapy accommodating space and a power supply accommodating space, the aromatherapy accommodating space accommodates the aromatherapy structure, and the power supply accommodating space accommodates the power supply.

Further, the light-emitting device has a ring shape, and the decoration member corresponds to a center of the ring shape.

Further, the light-emitting device is a sound-sensitive lamp.

Further, the control assembly includes a button, and the button is provided on the housing and controls the light-emitting device to emit light.

Further, the light-emitting device includes a lamp board and a lampshade, a groove is provided on the housing, and the lamp board and the lampshade are respectively mounted on the groove.

In the described vehicle-mounted device, both a light-emitting device and a cartoon device are provided on the housing of the vehicle-mounted device, so that esthetic experience of a user can be increased, and the vehicle-mounted device is more esthetically pleasing when being decorated in the environment inside a vehicle; in addition, the light-emitting device is provided as a sound-sensitive lamp, so that the light-emitting effects are converted according to the sound in the vehicle during use, the use effect is better and the interestingness is stronger.

### BRIEF DESCRIPTION OF THE DRAWINGS

The accompanying drawings are only used for exemplary description and should not be construed as limiting the present disclosure, and the same reference numerals are used for members having the same structure and function. In the drawings:

FIG. 1 is a schematic exploded structural diagram of a vehicle-mounted device provided by an embodiment of the present application;

FIG. 2 is a schematic exploded structural diagram of a vehicle-mounted device provided by a first embodiment of the present application;

FIG. 3 is a schematic structural diagram of a first aromatherapy frame in FIG. 2;

FIG. 4 is a schematic exploded structural diagram of a vehicle-mounted device provided by a second embodiment of the present application;

FIG. 5 is a schematic structural diagram of another embodiment of the aromatherapy storage compartment in FIG. 4;

FIG. 6 is a schematic exploded structural diagram of a vehicle-mounted device provided by a third embodiment of the present application;

FIG. 7 is a schematic structural diagram of another embodiment of the vehicle-mounted device in FIG. 6;

FIG. 8 is a schematic exploded structural diagram of a vehicle-mounted device provided by a fourth embodiment of the present application; and

FIG. 9 is a schematic structural diagram of another embodiment of the vehicle-mounted device in FIG. 8.

Description of reference signs: decoration assembly—1; housing—2; accommodating space—8; power supply accommodating space—81; aromatherapy accommodating space—82; ventilation channel—6; quick-release mechanism—7; power supply—3; aromatherapy structure—4; fixing device—5; decoration member—11; light-emitting device—12; control assembly—13; first portion—21; second portion—22; aromatherapy member—41; aromatherapy frame—42; fixing member—51; protective member—52; first cartoon member—1a; first lampshade—4a; decoration lamp—5a; lower housing—10a; upper housing—2a; magnet—6a; first power supply—3a; first aromatherapy piece—9a; first aromatherapy frame—8a; mounting column—7a; first fixing member—11a; first protective member—12a; first decoration member—1b; second lampshade—4b; first lamp board—7b; first button—6b; mounting base—9b; first surface cover—2b; cover plate—5b; panel—21b; second power supply—8b; aromatherapy material—3b; second fixing member—10b; second protective member—11b; second cartoon member—1c; third lampshade—5c; second lamp board—6c; second button—4c; first housing—7c; second surface cover—2c; rear cover/aromatherapy cover—9c; third power supply—3c; second aromatherapy piece—8c, third fixing member—10c, third protective member—11c, cartoon device—9d, fourth lampshade—1d, third lamp board—4d; control switch—3d; second housing—5d; cover body—8d; fourth power supply—2d; fixed pin—6d; and fourth protective member—7d.

### DETAILED DESCRIPTION

The following describes the technical solutions in the embodiments of the present application with reference to the accompanying drawings in the embodiments of the present application. In the description of the embodiments of the present application, unless otherwise specified, “/” indicates or. For example, A/B may indicate A or B; “and/or” herein is only an association relationship for describing associated objects, and indicates that three relationships may exist. For example, A and/or B may indicate three cases: A exists separately, both A and B exist, and B exists separately. In addition, in the description of the embodiments of the present application, “a plurality of” means two or more than two.

Hereinafter, the terms “first” and “second” are only used for descriptive purposes, and cannot be understood as indicating or implying relative importance or implicitly indicating the number of indicated technical features. Thus, the features defined by “first” and “second” may explicitly or implicitly include one or more of the features.

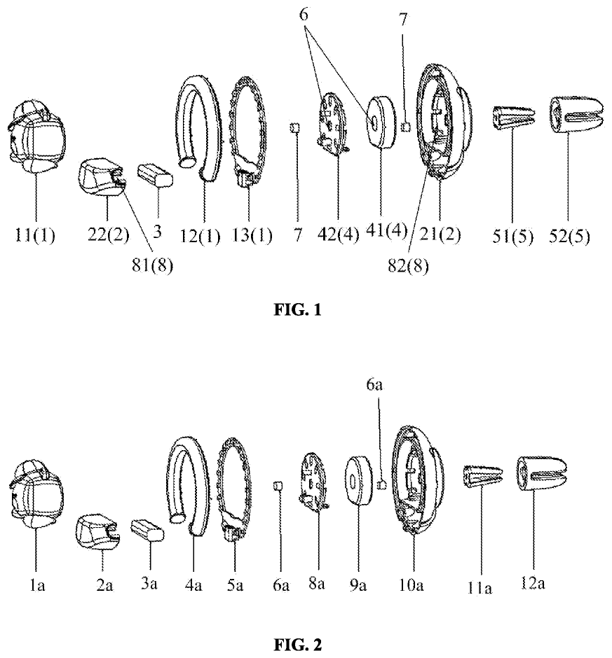

Please refer to FIG. 1, in this embodiment, exemplarily, an embodiment of the present application provides a vehicle-mounted device, including a housing 2 and a decoration assembly 1; the decoration assembly 1 includes a light-emitting device 12 and a decoration member 11, the decoration member 11 is detachably provided on the housing 2, and the light-emitting device 12 is provided outside the housing 2.

Optionally, the vehicle-mounted device further includes an aromatherapy structure 4, and the aromatherapy structure 4 is replaceably provided on the housing 2.

Optionally, the vehicle mounted device further includes a power supply 3, and the power supply 3 is replaceably provided in the housing 2 and supplies power to the light-emitting device 12.

Optionally, the decoration assembly 1 further includes a control assembly 13, both the control assembly 13 and the power supply 3 are electrically connected to the light-emitting device 12 respectively, and the control assembly 13 is provided on the housing 2 and controls the light-emitting device 12 to emit light.

Optionally, the vehicle-mounted device further includes a fixing device 5, and the fixing device 5 is provided on one side of the housing 2.

Optionally, the fixing device 5 includes a fixing member 51 and a protective member 52, the fixing member 51 is provided on the housing 2, and the protective member 52 is provided on the fixing member 51.

Optionally, the fixing member 51 is any one of a pin, a clamping claw, and a magnetic fixing device.

Optionally, the aromatherapy structure 4 includes an aromatherapy frame 42 and an aromatherapy member 41, and the aromatherapy member 41 is mounted on the aromatherapy frame 42.

Optionally, the vehicle-mounted device further includes a quick-release mechanism 7, and the quick-release mechanism 7 is detachably connected to the housing 2.

Optionally, the aromatherapy structure 4 is mounted on the quick-release mechanism 7.

Optionally, the decoration member 11 is mounted on the quick-release mechanism 7.

Optionally, the housing 2 includes a first portion 21 and a second portion 22, the second portion 22 is detachably mounted on the first portion 21, the decoration assembly 1 includes a light-emitting device 12 and a decoration member 11, the light-emitting device 12 is provided on the first portion 21, and the decoration member 11 is provided on the second portion 22.

Optionally, the vehicle-mounted device further includes an accommodating space 8, and the accommodating space 8 is provided on the housing 2 or the decoration member 11.

Optionally, the vehicle-mounted device further includes an accommodating space 8, and the accommodating space 8 is provided on the first portion 21 or the second portion 22.

Optionally, the vehicle-mounted device further includes a ventilation channel 6, and the ventilation channel 6 is provided on the housing 2 and connects the accommodating space 8 to an external environment.

Optionally, the vehicle-mounted device further includes an aromatherapy accommodating space 82 and a power supply accommodating space 81, the aromatherapy accommodating space 82 accommodates the aromatherapy structure 4, and the power supply accommodating space 81 accommodates the power supply 3.

Optionally, the light-emitting device 12 has a ring shape, and the decoration member 11 corresponds to a center of the ring shape.

Optionally, the light-emitting device 12 is a sound-sensitive lamp.

Optionally, the control assembly includes a button, and the button is provided on the housing and controls the light-emitting device to emit light.

Optionally, the light-emitting device 12 includes a lamp board and a lampshade, a groove is provided on the housing 2, and the lamp board and the lampshade are respectively mounted on the groove.

In the described vehicle-mounted device, both a light-emitting device and a cartoon device are provided on the housing of the vehicle-mounted device, so that esthetic experience of a user can be increased, and the vehicle-mounted device is more esthetically pleasing when being decorated in the environment inside a vehicle; in addition, the light-emitting device is provided as a sound-sensitive lamp, so that the light-emitting effects are converted according to the sound in the vehicle during use, the use effect is better and the interestingness is stronger.

In order to more specifically describe the technical solution provided by this embodiment, four specific embodiments are provided below.

### Specific Embodiment 1

Please refer to FIGS. 2-3, in this embodiment, specifically, the decoration member 11 specifically includes a first cartoon member 1a; the light-emitting device 12 specifically includes a first lampshade 4a and a decoration lamp 5a; the control assembly 13 specifically includes an electronic control mounting base and a decoration lamp switch; the first portion 21 specifically includes a lower housing 10a, and the second portion 22 specifically includes an upper housing 2a; the accommodating space 8 specifically includes an aromatherapy placement chamber; the ventilation channel 6 specifically includes ventilation holes or is hollow, etc., but is not limited thereto; the quick-release mechanism 7 specifically includes a magnet 6a; the power supply 3 specifically includes a first power supply 3a; the aromatherapy member 41 specifically includes a first aromatherapy piece 9a; the aromatherapy frame 42 specifically includes a first aromatherapy frame 8a; the fixing member 51 specifically includes a first fixing member 11a; and the protective member 52 specifically includes a first protective member 12a.

Specifically, the first aromatherapy frame 8a is provided inside the housing, and has an aromatherapy function without affecting the design of the appearance; the fixing device is provided on the housing; the vehicle-mounted device in the present technical solution can be fixed on the vehicle body by means of the fixing device, and can also be placed at the air outlet of the air conditioner or other positions of the vehicle body; the fixing device can be provided to be any one of a pin, a clamping jaw and a magnetic fixing device according to specific situations. In addition, in order to improve the aesthetics of the vehicle-mounted device, the decoration member includes a decoration lamp 5a and a first cartoon member 1a, the first cartoon member 1a is detachably provided on the housing by means of a first aromatherapy frame 8a, and various cartoon or doll images can be mounted and replaced according to the requirements of different consumer groups; and the decoration lamp Sa is fixedly provided on the housing, so that the appearance of the first cartoon member 1a can be improved by means of a light beam.

As shown in FIG. 1, the housing includes an upper housing 2a and a lower housing 10a which are detachably connected to each other; a first power supply 3a is provided within the upper housing 2a; and a first aromatherapy frame 8a is provided within the lower housing 10a by means of a quick-release mechanism. An annular mounting groove is provided on the lower housing 10a, the decoration lamp 5a is provided in the mounting groove, a first lampshade 4a is provided on the mounting groove for protecting the decoration lamp 5a, and the lighting effect of the decoration lamp 5a can be increased by designing the first lampshade 4a; the first aromatherapy frame 8a is provided at the central position of the lower housing 10a, and is located at the lower middle portion of the mounting groove. As the first cartoon member 1a is mounted on the first aromatherapy frame 8a, the first aromatherapy frame 8a is recessed in the middle of the lower housing 10a, the stereoscopic sense of the vehicle-mounted device can be increased in the vertical direction, and the aromatherapy can be hidden within the lower housing 10a, so that the first cartoon member 1a and the decoration lamp 5a reflect the glow alternately.

In the present technical solution, for more stable fixing, especially when the first fixing member 11a is fixed by means of a pin, the first protective member 12a is provided outside the pin so as to ensure that the vehicle-mounted device does not fall off when the vehicle body is shaken; and the first protective member 12a is provided, so that the vehicle body can be protected from being scratched by the pin, specifically, the first fixing member 11a of the pin is provided on the lower housing 10a and is located at the bottom of the first aromatherapy frame 8a.

In order to ensure that the first cartoon member 1a is detachably provided on the first aromatherapy frame 8a, and can be connected to the first aromatherapy frame 8a by means of an adhesive, a clamping mechanism or an integral connection mechanism, a combination manner of the adhesive and the clamping mechanism can be used, and specifically, a fixing column is provided on an upper surface of the first aromatherapy frame 8a, a mounting groove fitted with the fixing column is provided at the lower portion of the first cartoon member 1a, and the fixing column is connected to the mounting groove in a snap-fitting manner, and the adhesive can also be applied on the first aromatherapy frame 8a and the upper mounting surface of the first cartoon member 1a so as to assist the fixing therebetween; of course, the first cartoon member 1a and the first aromatherapy frame 8a can also be integrally formed through injection molding, and can be integrally replaced when being replaced.

In the technical solution, in order to conveniently fix the first aromatherapy piece 9a, an aromatherapy placement chamber is provided between the first aromatherapy frame 8a and the housing, then the first aromatherapy piece 9a can be placed within the chamber, and several ventilation holes are provided on the aromatherapy placement chamber or a side wall of the aromatherapy placement chamber has a hollow structure, thereby ensuring that the aromatherapy placement chamber can be in communication with the outside air, and thus releasing the aroma of the first aromatherapy piece 9a.

In addition, optimized structural layout is that the upper housing 2a is located at the front portion of the lower housing 10a, a device lamp electronic control mounting base is provided on the mounting groove corresponding to the mounting position of the upper housing 2a, the switch of a decoration lamp 5a is provided on the upper housing 2a, and the volume of the upper housing 2a is relatively small relative to the first lampshade 4a of the device, so that the decorative effects can be increased to a greater extent; specifically, the decoration lamp 5a is a music-sensitive lamp, and can perform light adjustment according to music in the vehicle, thereby being more interesting.

The quick-release mechanism between the first aromatherapy frame 8a and the housing is an attraction mechanism of a magnet 6a or a plug-in mechanism. When the quick-release mechanism is an attraction mechanism of the magnet 6a, a mounting column 7a is provided at the bottom of the first aromatherapy frame 8a, a fixing portion is provided at the middle portion of the lower housing 10a, the mounting column 7a and the fixing portion are respectively provided with magnets 6a fitted with each other, and the first aromatherapy piece 9a is fixed on the mounting column 7a during use, and then is directly attracted onto the magnet 6a on the fixing portion of the lower housing 10a through the magnet 6a on the mounting column 7a. When the first aromatherapy frame 8a is connected to the lower housing 10a by means of a plug-in mechanism, a mounting column 7a and a mounting groove structure which cooperate with each other can be provided. Of course, the technical solution of the present application is not limited to the two quick-release mechanisms mentioned above.

### Specific Embodiment 2

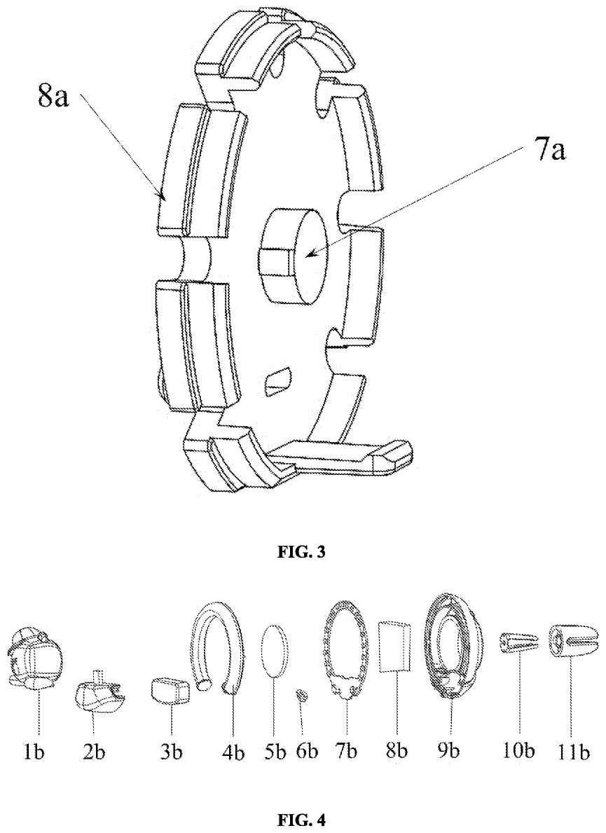

Please refer to FIGS. 4 to 5, in this embodiment, specifically, the decoration member 11 specifically includes a first decoration member 1b; the light-emitting device 12 specifically includes a second lampshade 4b and a first lamp board 7b; the control assembly 13 specifically includes a first button 6b; the first portion 21 specifically includes a mounting base 9b, and the second portion 22 specifically includes a first surface cover 2b and a cover plate 5b; the accommodating space 8 specifically includes an accommodating cavity; the power supply accommodating space 81 specifically includes a power supply accommodating chamber; the aromatherapy accommodating space 82 specifically includes an aromatherapy accommodating chamber; the ventilation channel 6 specifically includes ventilation holes or is hollow, etc., but is not limited thereto; the quick-release mechanism 7 specifically includes a panel 21b; the power supply 3 specifically includes a second power supply 8b; the aromatherapy member 41 specifically includes an aromatherapy material 3b; the fixing member 51 specifically includes a second fixing member 10b, and the protective member 52 specifically includes a second protective member 11b.

Specifically, an accommodating cavity for placing the second power supply 8b and the aromatherapy material 3b is further provided within the housing. The decoration assembly and the aromatherapy device are integrated, thereby improving the appearance of the vehicle-mounted decoration. In the solution of the present application, the accommodating cavity for accommodating the power supply and the aromatherapy can be provided integrally or separately; preferably, the separate arrangement of the accommodating cavity specifically includes a power supply placement chamber and an aromatherapy placement chamber, the power supply placement chamber is provided inside the housing, the aromatherapy placement chamber is provided at the front portion of the housing. As the replacement frequency of the battery is lower than that of the aromatherapy, and in order to reduce the interference of external factors (such as water) on the power supply, the power supply placement chamber is provided inside the housing to be better protected, and the aromatherapy placement chamber is provided at the front portion or outside the housing, facilitating the diffusion of fragrance and facilitating opening for adding the aromatherapy material 3b.

As shown in FIG. 3, for the described preferred embodiment, the housing includes a mounting base 9b, a cover plate 5b and a first surface cover 2b, the power supply placement chamber is provided in the middle of the mounting base 9b, and the cover plate 5b covers an opening of the mounting base 9b corresponding to the power supply placement chamber so that the power supply placement chamber forms an enclosed space; the first surface cover 2b is provided on the mounting base 9b, the aromatherapy placement chamber is provided in the first surface cover 2b, and the cover is designed to enable the aromatherapy device to be provided independently, facilitating replacement.

About the design of the integrated decoration lamp in the present technical solution, the specific mounting method is as follows: an annular mounting groove is provided on the mounting base 9b, a first lamp board 7b of a decoration lamp is provided in a mounting groove, and a light-transmitting second lampshade 4b is provided on the mounting groove, so that an annular lamp strip can be formed on the mounting base 9b. As shown in FIG. 3, a mounting notch of the first surface cover 2b is preferably provided below the lamp strip, so that the first decoration member 1b can be positioned in the center of the annular light strip when being mounted on the first surface cover 2b so as to increase the lighting effect; of course, the light strip can also be provided as an annular shape, and the first surface cover 2b can be provided on the cover plate 5b or directly connected to the power supply placement chamber, which is the integrated arrangement solution of the accommodating chamber mentioned above.

In the technical solution of the present application, the first decoration member 1b is detachably provided on the first surface cover 2b. One mounting method is that the first decoration member 1b is directly and fixedly connected to an upper side wall of the first surface cover 2b as shown in FIG. 3, openings for replacing the aromatherapy material are provided on other sides of the first surface cover 2b, such as the lower portion, the front portion, or the left and right side walls; likewise, preferably, an opening is provided at a side close to the mounting base 9b, and the surface cover is detachably provided on the mounting base, then when the aromatherapy material needs to be replaced, the surface cover can be directly removed; the first decoration member 1b and the first surface cover 2b can be fixed by bonding, clamping, or any of the connection methods through the fixed column. Another mounting method is as shown in FIG. 4, a detachable panel 21b is provided on the first surface cover 2b, the first decoration member 1b is fixedly provided on the panel 21b, and in this embodiment, the opening direction of the panel can be provided upwards or forwards; an aromatherapy opening is provided on a side wall of the first surface cover 2b or a part of the first surface cover 2b is provided in a hollowed-out manner; and in an alternative technical solution, a decoration member can be provided on an upper side face of a surface cover, then a detachable panel is provided on a lower side face of the surface cover, and when in use, the aromatherapy material 3b can be replaced by the panel on the lower side. In addition, the first decoration member 1b can be detachably mounted on the first surface cover 2b, allowing for the mounting and replacement of various cartoon or doll images according to the requirements of different consumer groups.

As a further optimization of the technical solution of the present application, the vehicle-mounted device also includes a fixing device, the fixing device is provided on the mounting base 9b and located in a position opposite to the first surface cover 2b. The fixing device includes a second fixing member 10b and a second protective member 11b, the second fixing member 10b is provided on the mounting base 9b, and the second protective member 11b is provided on the second fixing member 10b. The second fixing member 10b is any one of a pin, a clamping claw and a magnetic fixing device. The second protective member 11b is provided outside the pin so as to ensure that the vehicle-mounted device does not fall out when the vehicle body is shaken; and the second protective member 11b is provided, so that the vehicle body can be protected from being scratched by the pin, specifically, the second fixing member 10b is provided on the mounting base 9b and is located at the bottom of the power supply placement chamber.

The decoration lamp in the described technical solution further includes a first button 6b for controlling the operation of the decoration lamp, and the first button 6b is provided on the mounting base 9b; a preferable decoration lamp is a music-sensitive lamp. By playing music in a vehicle, the color of a lamp strip can be changed, so that the lamp strip is more ornamental.

### Specific Embodiment 3

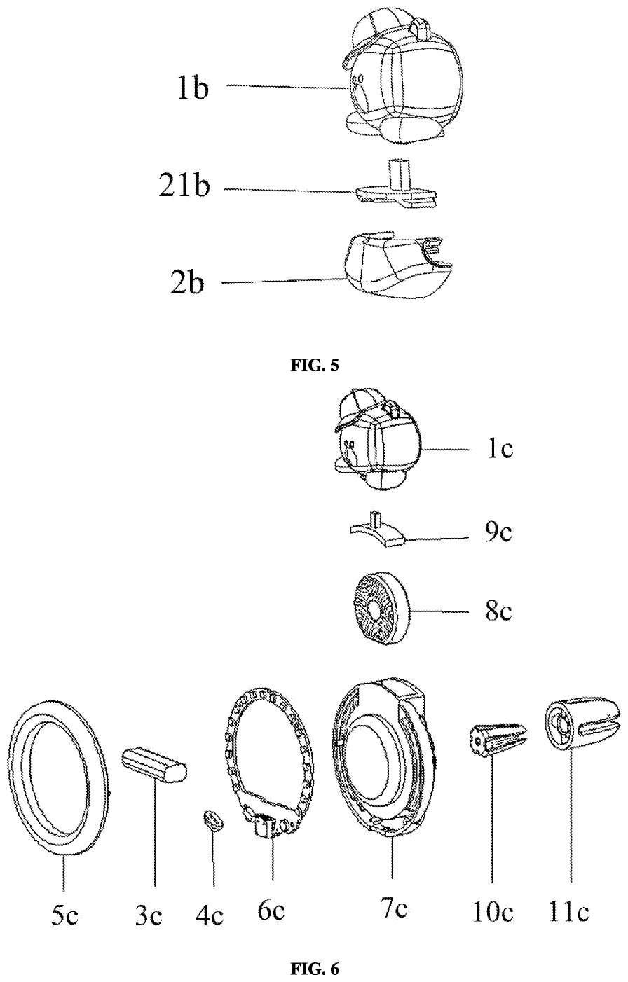

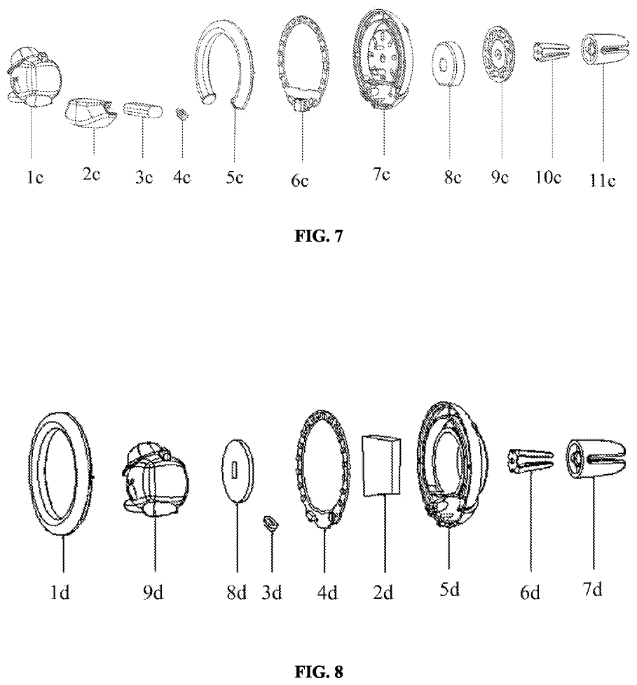

Please refer to FIGS. 6 to 7, in this embodiment, specifically, the decoration member 11 specifically includes a second cartoon member 1c; the light-emitting device 12 specifically includes a third lampshade 5c and a second lamp board 6c; the control assembly 13 specifically includes a second button 4c; the first portion 21 specifically includes a first housing 7c, and the second portion 22 specifically includes a second surface cover 2c; the accommodating space 8 specifically includes an aromatherapy accommodating cavity; the quick-release mechanism 7 specifically includes a rear cover 9c or an aromatherapy cover 9c; the power supply 3 specifically includes a third power supply 3c; the aromatherapy member 41 specifically includes a second aromatherapy piece 8c; the aromatherapy frame 42 specifically includes a second aromatherapy frame; the fixing member 51 specifically includes a third fixing member 10c; and the protective member 52 specifically includes a third protective member 11c.

Specifically, the decoration lamp and the second cartoon member 1c are respectively provided outside the first housing 7c, the second cartoon member 1c can be mapped by the decoration lamp, so that the second cartoon member has better appearance; in addition, the aromatherapy assembly is provided within the first housing 7c, better achieving the aromatherapy function.

As shown in FIGS. 1 and 2, the decoration lamp includes a second lamp board 6c, a third lampshade 5c, a third power supply 3c and a second button 4c, a groove is provided on the first housing 7c, and the second lamp board 6c and the third lampshade 5c are respectively mounted within the groove, the second button 4c is provided on a side wall of the first housing 7c for controlling the operation of the second lamp board 6c. Specifically, the third lampshade 5c can be provided in an annular shape as shown in FIG. 1, and then the third power supply 3c is provided at a middle position of the first housing 7c, the placement chamber of the third power supply 3c can be separately provided, and the third power supply can share the same chamber with the aromatherapy accommodating cavity. Another embodiment is to design the third lampshade 5c as an annular lamp strip having a certain notch as shown in FIG. 2, and then provide a placement chamber of a third power supply 3c at the notch, and is mounted on the first housing 7c through the second surface cover 2c, and at this time, there are more design options for the second cartoon member 1c. In the solution of the present application, the preferred decoration lamp is a music-sensitive light. By playing music in a vehicle, the color of light can be changed, so that the lamp strip is more ornamental, making it more esthetically pleasing.

About the design of the aromatherapy assembly, in the technical solution, an aromatherapy accommodating cavity is provided within the first housing 7c, and the second cartoon member 1c is fixedly provided on the first housing 7c of a side wall of the aromatherapy accommodating cavity. About the embodiment of the design, it is only required that the second cartoon member 1c is in a position avoiding the mounting of the fixing assembly. Specifically, the technical solution of the present application provides the following two preferred embodiments: one is as shown in FIG. 1, an aromatherapy cover 9c for replacing an aromatherapy is provided on the first housing 7c corresponding to the side wall of the aromatherapy accommodating cavity, and the second cartoon member 1c is fixed on the aromatherapy cover 9c. Specifically, the aromatherapy cover 9c is provided on the first housing 7c at the upper portion of the accommodating cavity, and a fixing column is provided on the aromatherapy cover 9c, the second cartoon member 1c is mounted on the fixing column by means of an adhesive, clamping, riveting or screwing, the aromatherapy cover 9c is fixed above the first housing 7c corresponding to the aromatherapy accommodating cavity in a snap-fitting manner; when in use, the aromatherapy cover 9c can be directly opened by the second cartoon member 1c, and then the second aromatherapy piece 8c is replaced, being convenient to use; the second cartoon member 1c is provided at the upper portion, having a stronger decorative effect. In the second embodiment, as shown in FIG. 2, the second cartoon member 1c is mounted directly in front of the first housing 7c, opposite to the fixing assembly, specifically mounted at a central position of the annular lamp strip, and preferably cooperates therewith to provide a rear cover 9c at the rear of the aromatherapy accommodating cavity, then the third fixing member 10c of the fixing assembly is mounted on the rear cover 9c, and the rear cover 9c is connected to the first housing 7c corresponding to the aromatherapy accommodating cavity by means of threads, clamping or screws; when replacing the aromatherapy, the aromatherapy accommodating cavity can be replaced by opening the aromatherapy accommodating cavity by means of the third fixing member 10c. Of course, on the basis of the above two embodiments, the second cartoon member 1c may also be mounted directly in front of the first housing 7c, then an aromatherapy cover is provided on the housing corresponding to the upper side wall of the aromatherapy accommodating cavity, and the aromatherapy material is replaced by opening the aromatherapy cover separately.

The vehicle-mounted device in the foregoing embodiment further includes a fixing assembly. The fixing assembly preferably includes a third fixing member 10c and a third protective member 11c, the third fixing member 10c is provided on the lower first housing 7c, and the third protective member 11c is provided at a fixed position on the third fixing member 10c for protecting the vehicle body. The third fixing member 10c is any one of a pin, a clamping claw, and a magnetic fixing assembly.

According to a preferred technical solution, the aromatherapy assembly includes a second aromatherapy piece 8c and a second aromatherapy frame, the second aromatherapy piece 8c is provided within the aromatherapy accommodating cavity by means of the second aromatherapy frame, so that the second aromatherapy piece 8c can be conveniently fixed. Specifically, when the second aromatherapy piece 8c or the aromatherapy paste is replaced, the second aromatherapy frame is taken out, and then the second aromatherapy frame is mounted back after being externally mounted.

### Specific Embodiment 4

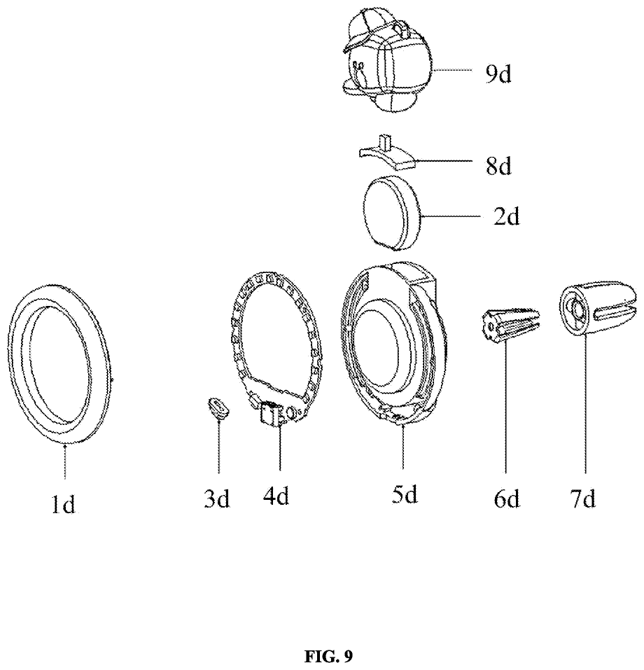

Please refer to FIGS. 8 to 9, in this embodiment, specifically, the decoration member 11 specifically includes a cartoon device 9d; the light-emitting device 12 specifically includes a fourth lampshade 1d and a third lamp board 4d; the control assembly 13 specifically includes a control switch 3d; the first portion 21 specifically includes a second housing 5d, and the second portion 22 specifically includes a cover 8d; the accommodating space 8 specifically includes a first storage chamber and a second storage chamber; the power supply 3 specifically includes a fourth power supply 2d; the fixing member 51 specifically includes a fixing pin 6d; and the protective member 52 specifically includes a fourth protective member 7d.

Specifically, the decoration assembly can be fixed on the second housing 5d by means of a screw structure or a snap-fit structure; the light-emitting device is preferably provided as a sound-sensitive lamp. By designing the sound-sensitive lamp, light can be converted according to music or other sounds in a vehicle during driving, so that the vehicle-mounted device of the present application is more ornamental. In addition, in order to facilitate the fixing of the vehicle-mounted device in the present application, a 3M adhesive or a fixing pin 6d is provided at the rear portion or lower portion of the second housing 5d; and when the fixing pin 6d is provided, a fourth protective member 7d is correspondingly provided on the fixing pin 6d, and is preferably a rubber protective sleeve and can be conveniently placed at the front portion of the automobile or fixed at the air outlet of the air conditioner by providing a fixing mechanism. The specific implementations are explained as follows. In addition, the cartoon device 9d of the present application can be a cartoon doll or other cartoon designs.

Implementation 1: as shown in FIG. 1, the light-emitting device includes a third lamp board 4d, a fourth lampshade 1d, a fourth power supply 2d and a control switch 3d; the third lamp board 4d is provided inside the fourth lampshade 1d, the fourth lampshade 1d is fixed on the second housing 5d, the fourth power supply 2d and the control switch 3d are electrically connected to the third lamp board 4d respectively, and the fourth power supply 2d is preferably a coin cell battery or a rechargeable fourth power supply 2d. The fourth lampshade 1d is provided, so that the use experience of the third lamp board 4d during operation can be increased without giving users a dazzling feeling. In addition, the fourth lampshade 1d is detachably provided on the second housing 5d, and the fourth lampshade 1d can be designed as a plurality of theme colors, so that the diversity of production can be increased, and more choices can be provided for a consumer. Specifically, the fourth lampshade 1d can be provided to be milk white, light yellow, light green, pink, etc., the fourth lampshade 1d can enhance the overall lighting atmosphere inside the vehicle, and the color of the LED lamp bead on the matching third lamp board 4d changes, making the entire vehicle-mounted device more interesting and viewable.

In this implementation, a first storage chamber is provided within the second housing 5d, the fourth power supply 2d is provided in the first storage chamber, the control switch 3d is provided at the lower portion of the first storage chamber. Specifically, the first storage chamber is provided at the middle portion of the second housing 5d, a cover body 8d is provided on the first placement chamber, and the cover body 8d can be provided at the front portion or the upper portion of the first placement chamber, so that the battery in the first storage chamber can be replaced conveniently.

In addition, in this implementation, the fourth lampshade Id is annular and located at the front port of the second housing 5d; the cartoon device 9d is provided in the middle portion of the annular fourth lampshade 1d; and the cartoon device 9d is detachably provided on the cover body 8d of the first storage chamber, or when the cover body 8d is provided at the upper portion of the first storage chamber, the cartoon device 9d is directly fixed at the front portion of the second housing 5d. In this implementation, the light-emitting device surrounds the circumference of the cartoon device 9d, and can enhance decorative effects of the cartoon device 9d.

Implementation 2: on the basis of implementation 1, a second storage chamber is provided in the cartoon device 9d, and the fourth power supply 2d is provided in the second storage chamber. Correspondingly, the fourth lampshade 1d and the third lamp board 4d can be provided in an annular structure having a notch at the bottom, and the control switch 3d is provided at the lower portion of the cartoon device 9d and is correspondingly provided at the notch of the fourth lampshade 1d, thereby facilitating the control of the light-emitting device. In this implementation, the first storage chamber can be reserved, and a standby cartoon device 9d can be placed in the first storage chamber, thereby bringing more usage options to consumers.

In this implementation, the arrangement positions of the light-emitting device and the decoration device are the same as those in implementation 1, i.e., the light-emitting device is provided at the front of the second housing 5d, and the cartoon device 9d is provided at the front of the second housing 5d.

Implementation 3: on the basis of implementation 1 and implementation 2, in this implementation, the light-emitting device is provided at the front of the second housing 5d, and the cartoon device 9d is provided on the upper portion of the second housing 5d; and in this implementation, the structures of the fourth lampshade 1d and the third lamp board 4d are in an annular layout, can be provided to be in other shapes such as an X shape and a radiation shape with an ornamental appearance, so as to provide more diversified designs for the vehicle-mounted device of the present application and provide consumers with more choices, so that a customer can select a their preferred and relatively unique vehicle-mounted device to enhance the beauty of the vehicle.

Implementation 4: on the basis of the above implementations, in this implementation, the light-emitting device is provided inside the cartoon device 9d, and the cartoon device 9d is provided above or in front of the second housing 5d. This can further enhance the decorative effects of the cartoon device 9d.

In the description of this embodiment, unless otherwise specified, the meaning of “a plurality of” is two or more. The foregoing content is merely specific embodiments of the present application, but the scope of protection of the present application is not limited thereto. Any change or replacement within the technical scope disclosed in the present application shall belong to the scope of protection of the present application. Therefore, the scope of protection of the present application shall be subject to the scope of protection of the claims.

What is claimed is:

1. A vehicle-mounted device, comprising a housing and a decoration assembly, wherein the decoration assembly comprises a light-emitting device and a decoration member, the decoration member is detachably provided on the housing, and the light-emitting device is provided outside the housing.

2. The vehicle-mounted device according to claim 1, further comprising an aromatherapy structure, wherein the aromatherapy structure is replaceably provided on the housing.

3. The vehicle mounted device according to claim 1, further comprising a power supply, wherein the power supply is replaceably provided in the housing and supplies power to the light-emitting device.

4. The vehicle-mounted device according to claim 1, wherein the decoration assembly further comprises a control assembly, both the control assembly and the power supply are electrically connected to the light-emitting device, and the control assembly is provided on the housing and controls the light-emitting device to emit light.

5. The vehicle-mounted device according to claim 1, further comprising a fixing device, wherein the fixing device is provided on one side of the housing.

6. The vehicle-mounted device according to claim 5, wherein the fixing device comprises a fixing member and a protective member, the fixing member is provided on the housing, and the protective member is provided on the fixing member.

7. The vehicle-mounted device according to claim 6, wherein the fixing member is any one of a pin, a clamping claw, and a magnetic fixing device.

8. The vehicle-mounted device according to claim 2, wherein the aromatherapy structure comprises an aromatherapy frame and an aromatherapy member, and the aromatherapy member is mounted on the aromatherapy frame.

9. The vehicle-mounted device according to claim 1, further comprising a quick-release mechanism, wherein the quick-release mechanism is detachably connected to the housing.

10. The vehicle-mounted device according to claim 9, wherein the aromatherapy structure is mounted on the quick-release mechanism.

11. The vehicle-mounted device according to claim 9, wherein the decoration member is mounted on the quick-release mechanism.

12. The vehicle-mounted device according to claim 1, wherein the housing comprises a first portion and a second portion, the second portion is detachably mounted on the first portion, the decoration assembly comprises the light-emitting device and the decoration member, the light-emitting device is provided on the first portion, and the decoration member is provided on the second portion.

13. The vehicle-mounted device according to claim 1, further comprising an accommodating space, wherein the accommodating space is provided on the housing or the decoration member.

14. The vehicle-mounted device according to claim 12, further comprising an accommodating space, wherein the accommodating space is provided on the first portion or the second portion.

15. The vehicle-mounted device according to claim 13, further comprising a ventilation channel, wherein the ventilation channel is provided on the housing and connects the accommodating space to an external environment.

16. The vehicle-mounted device according to claim 1, further comprising an aromatherapy accommodating space and a power supply accommodating space, wherein the aromatherapy accommodating space accommodates the aromatherapy structure, and the power supply accommodating space accommodates the power supply.

17. The vehicle-mounted device according to claim 14, wherein the light-emitting device has a ring shape, and the decoration member corresponds to a center of the ring shape.

18. The vehicle-mounted device according to claim 1, wherein the light-emitting device is a sound-sensitive lamp.

19. The vehicle-mounted device according to claim 4, wherein the control assembly comprises a button, and the button is provided on the housing and controls the light-emitting device to emit light.

20. The vehicle-mounted device according to claim 1, wherein the light-emitting device comprises a lamp board and a lampshade, a groove is provided on the housing, and the lamp board and the lampshade are respectively mounted on the groove.
